(19) United States
(12) Patent Application Publication
(10) Pub. No.: US 2005/0020922 A1
(43) Pub. Date: Jan. 27, 2005

(54) Materials and methods for near-infrared and infrared intravascular imaging

(76) Inventors: John V. Frangioni, Wayland, MA (US); Moungi G. Bawendi, Boston, MA (US); Sungjee Kim, Pasadena, CA (US); Yong Taik Lim, Cheonju (KR)

Correspondence Address: STEPTOE & JOHNSON LLP, 1330 CONNECTICUT AVENUE, N.W., WASHINGTON, DC, 20036, US

(21) Appl. No.: 10/772,424

(22) Filed: Feb. 6, 2004

Related U.S. Application Data
(60) Provisional application No. 60/451,247, filed on Mar. 4, 2003.

(51) Int. Cl. A61B 6/00

(52) U.S. Cl. 600/473; 257/82

(57) ABSTRACT

Vasculature can be imaged with emissive semiconductor nanocrystals, for example, in the near infrared.

### CLAIM OF PRIORITY

This application claims priority under 35 U.S.C. § 119(e) to U.S. patent application Ser. No. 60/451,247, filed on Mar. 4, 2003, the entire contents of which are hereby incorporated by reference.

### FEDERALLY SPONSORED RESEARCH OR DEVELOPMENT

The U.S. Government may have certain rights in this invention pursuant to Contract Nos. DMR-9808941 and DMR-0213282 by the Office of Naval Research, and Contract Nos. DE-FG02-01ER63188 and NIH R21 EB-00673 by the Department of Energy.

### TECHNICAL FIELD

The invention relates to imaging tissue and organs.

### BACKGROUND

Semiconductor nanocrystals (QDs) having small diameters can have properties intermediate between molecular and bulk forms of matter. For example, nanocrystals based on semiconductor materials having small diameters can exhibit quantum confinement of both the electron and hole in all three dimensions, which leads to an increase in the effective band gap of the material with decreasing crystallite size. Consequently, both the optical absorption and emission of nanocrystals shift to the blue (i.e., to higher energies) as the size of the crystallites decreases. Semiconductor nanocrystals can have a narrow fluorescence band whose emission wavelength is tunable with the size and material of the nanocrystals.

### SUMMARY

In general, imaging of vasculature can be performed by introducing emissive semiconductor nanocrystals into tissue. The emissive nanocrystals can emit irradiation in the near-infrared (NIR) or infrared wavelength region. The size of the nanocrystal and the coating on a surface of the nanocrystal can be selected to prolong imaging of the vasculature with high sensitivity. The imaging can be in vivo imaging.

Traditionally, in vivo near-infrared (NIR) fluorescence imaging of the vasculature is performed with low-molecular weight organic dyes such as indocyanine green (ICG) or the carboxylic acid of IRDye78 (IRDye78-CA). Unfortunately, these small fluorophores only provide short time windows for imaging (i.e., immediately after intravenous injection) since they rapid leak out of the vasculature and into tissue. Advantageously, emissive semiconductor nanocrystals of the appropriate size and coating permit provide prolonged imaging of the vasculature with high sensitivity. The nanocrystals are excited by light, and emit light, thereby replacing the need to produce images using X-ray technology. The size of NIR nanocrystals keeps particles in vasculature for prolonged imaging. The method allows for sensitive detection of normal tissue and tumor vascular during surgery, sensitive detection of sites of bleeding during surgery, and sensitive measurement of tissue perfusion during surgery and surgical repairs.

In one aspect, an imaging composition includes a semiconductor nanocrystal having an outer layer bonded to the nanocrystal.

In another aspect, a method of imaging tissue includes introducing a composition including a semiconductor nanocrystal into the tissue, and detecting emission from the semiconductor nanocrystal. The tissue can be vasculature. The emission can be in the near-infrared (NIR) or infrared wavelength region. Introducing the composition can include injecting the composition into a body, for example, into the vascular system of a body. Detecting emission can include monitoring tissue or tumor vascular during surgery, monitoring body sites of bleeding during surgery, or monitoring tissue perfusion during surgery and surgical repairs.

The semiconductor nanocrystal can have a diameter of between 5 nm and 10 nm. The outer layer can include a polydentate ligand. The nanocrystal can emit light having a wavelength greater than 700 nm. The nanocrystal can include a core of a first semiconductor material and an overcoating of a second semiconductor material on the core wherein the first semiconductor material and the second semiconductor material are selected so that, upon excitation, one carrier is substantially confined to the core and the other carrier is substantially confined to the overcoating.

Other features, objects, and advantages will be apparent from the description and drawings, and from the claims.

### DESCRIPTION OF DRAWINGS

FIG. 1 is a graph depicting photoproperties of 752 nm near-infrared nanocrystals.

FIGS. 2A-D are drawings depicting an experimental geometry and quantum dot performance in scattering and/or absorbing media and tissue.

FIGS. 3A-B are graphs depicting predicted photon transmission properties of biological tissue as a function of scatter, H2O to Hb ratio, and thickness.

FIG. 4 is a graph depicting predicted absorbance of NIR and IR semiconductor nanocrystals as a function of tissue scatter, H2O to Hb ratio, and thickness.

FIGS. 5A-B are graphs depicting comparison of NIR and IR semiconductor nanocrystal performance as a function of tissue scatter, H2O to Hb ratio, and thickness.

FIGS. 6A-B are graphs and photograph depicting NIR fluorescence imaging of the coronary vasculature using NIR semiconductor nanocrystal contrast agents.

### DETAILED DESCRIPTION

Fluorescent semiconductor nanocrystals are excellent contrast agents for biomedical assays and imaging. A unique property of semiconductor nanocrystals is that their absorbance increases with increasing separation between excitation and emission wavelengths. Much of the enthusiasm for using semiconductor nanocrystals in vivo stems from this property, since photon yield should be proportional to the integral of the broadband absorption. Tissue scatter and absorbance can sometimes offset increasing semiconductor nanocrystal absorption at bluer wavelengths, and counteract this potential advantage. By using a previously validated mathematical model, the effects of tissue absorbance, tissue scatter, wavelength dependence of the scatter, water to hemoglobin ratio, and tissue thickness on semiconductor nanocrystal performance were explored. When embedded in biological fluids and tissues, semiconductor nanocrystal excitation wavelengths can be quite constrained, and that excitation and emission wavelengths should be selected carefully based on the particular application. Near-infrared semiconductor nanocrystals optimized for imaging systems with white light excitation and a silicon CCD camera were produced and used to image the sentinel lymph node in real time. Emissive fluorescent semiconductor nanocrystal contrast agents optimized for specific biomedical applications. Other applications of semiconductor nanocrystals for imaging are described in co-pending application filed Mar. 4, 2003, entitled, “Materials and Methods for Near-Infrared and Infrared Lymph Node Mapping,” U.S. Ser. No. 60/451,246, which is incorporated by reference in its entirety.

Semiconductor nanocrystals are inorganic fluorophores that are currently being investigated for use as luminescent biological probes due to their nanometer dimensions and unique optical properties. Compared to conventional fluorophores and organic dyes, semiconductor nanocrystals have a number of attractive characteristics including high absorption cross-section, broadband absorption that increases at bluer wavelengths, relatively narrow and symmetric luminescence bands, simultaneous excitation of semiconductor nanocrystals with different emission wavelengths using a single excitation wavelength, and potentially high resistance to photo-degradation. Although the synthesis of semiconductor nanocrystals is performed in organic solvents, various surface chemistries can impart aqueous solubility and permit conjugation to biomolecules such as proteins, oligonucleotides, antibodies, and small molecule ligands. Such “targeted” semiconductor nanocrystals have been reported as contrast agents for nucleic acid hybridization, cellular imaging, immunoassays, and recently, tissue-specific homing in vivo. See, for example, Bruchez et al., Science281:2013-2016 (1998); Chan and Nie, Science 281:2016-2018 (1998); Mattoussi et al., J. Am. Chem. Soc. 122:12142-12150 (2000); Klarreich, Nature 413:450-452 (2001); Chan et al., Curr. Opin. Biotechnol. 13:40-46 (2002); Wu et al., “Immunofluorescent labeling of cancer marker Her2 and other cellular targets with semiconductor quantum dots,” Nature Biotechnol., published online Dec. 2, 2002 doi: 10.1038/nbt764; Dubertret et al., Science 298:1759-1762 (2002); Pathak et al., J. Am. Chem. Soc. 123:4103-4104 (2001); Gerion et al., J. Am. Chem. Soc. 124:7070-7074 (2002); Goldman et al., J. Am. Chem. Soc. 124:6378-6382 (2002); Goldman et al., Anal. Chem. 74:841-847 (2002); Rosenthal et al., J. of the Am. Chem. Soc. 124:4586-4594 (2002); Akerman et al., Proc. Natl. Acad. Sci. USA 99:12617-12621 (2002); and Jaiswal et al., “Long-term multiple color imaging of live cells using quantum dot bioconjugates,” Nature Biotechnol., published online Dec. 2, 2002 doi: 10.1038/nbt767, each of which is incorporated by reference in its entirety.

Another potential application of semiconductor nanocrystals is as fluorescent contrast agents for biomedical imaging. However, in vivo applications, and especially reflectance fluorescence imaging (the impetus for this study), require deep photon penetration into and out of tissue. In living tissue, total photon attenuation is the sum of attenuation due to absorbance and scatter. Scatter describes the deviation of a photon from the parallel axis of its path, and can occur when the tissue inhomogeneity is small relative to wavelength (Rayleigh-type scatter), or roughly on the order of wavelength (Mie-type scatter). For inhomogeneities at least ten times less than the wavelength, Rayleigh-type scatter is proportional to the reciprocal 4^{th} power of wavelength. In living tissue, photon scatter is the result of multiple scattering events, and in general terms can be considered either dependent on wavelength or independent of wavelength. For example, in rat skin, scatter is proportional to λ^{-2.8}, suggesting strong wavelength-dependence, however, in post-menopausal human breast, scatter is proportional to λ^{-0.6}, suggesting weak wavelength-dependence. See, for example, Zaheer et al., Nature Biotechnol. 19:1148-1154 (2001); Nakayama et al., “Functional near-infrared fluorescence imaging for cardiac surgery and targeted gene therapy,” Molecular Imaging (2002); Cheong et al., IEEE J. Quantum Electronics 26:2166-2195 (1990); and Cerussi et al., Acad. Radiol. 8:211-218 (2001), each of which is incorporated by reference in its entirety.

Given the relatively low absorbance and scatter of living tissue in the near-infrared (NIR; 700 nm to 1000 nm) region of the spectrum, considerable attention has focused on NIR fluorescence contrast agents. For example, conventional NIR fluorophores with peak emission between 700 nm and 800 nm have been used for in vivo imaging of protease activity, somatostatin receptors, sites of hydroxylapatite deposition, and myocardial vascularity, to name a few. To date, however, a systematic analysis of how tissue optical properties might affect semiconductor nanocrystal performance in vivo, and whether infrared (IR), rather than NIR, wavelengths could potentially improve overall photon yield, has not been presented. In this study, a mathematical model was used to predict how various tissue characteristics will affect semiconductor nanocrystal performance in vivo, and have used this model to select optimal semiconductor nanocrystal excitation and emission wavelengths for various imaging applications. Based on these results, a particular NIR semiconductor nanocrystal was synthesized and report the first use of NIR semiconductor nanocrystals for real-time in vivo vascular imaging. See, for example, Zaheer et al., Nature Biotechnol. 19:1148-1154 (2001); Nakayama et al., “Functional near-infrared fluorescence imaging for cardiac surgery and targeted gene therapy,” Molecular Imaging (2002); Weissleder, Nature Biotechnol. 19:316-7 (2001); Weissleder et al., Nature Biotechnol. 17:375-378 (1999); Becker et al., Nature Biotechnol. 19:327-31 (2001); and Bugaj et al., J. Biomed. Opt. 6:122-33 (2001); Gardner et al., Lasers Surg. Med 18:129-38 (1996), each of which is incorporated by reference in its entirety.

Nanocrystal cores can be prepared by the pyrolysis of organometallic precursors in hot coordinating agents. See, for example, Murray, C. B., et al., J. Am. Chem. Soc. 1993, 115, 8706, and Mikulec, F., Ph.D. Thesis, MIT, Cambridge, 1999, each of which is incorporated by reference in its entirety. Growth of shell layers on the bare nanocrystal cores can be carried out by simple modifications of conventional overcoating procedures. See, for example, Peng, X., et al., J. Am. Chem. Soc. 1997, 119, 7019, Dabbousi, B. O., et al., J. Phys. Chem. B 1997, 101, 9463, and Cao, Y. W. and Banin U., Angew. Chem. Int. Edit. 1999, 38, 3692, each of which is incorporated by reference in its entirety.

A coordinating agent can help control the growth of the nanocrystal. The coordinating agent is a compound having a donor lone pair that, for example, has a lone electron pair available to coordinate to a surface of the growing nanocrystal. Solvent coordination can stabilize the growing nanocrystal. Typical coordinating agents include alkyl phosphines, alkyl phosphine oxides, alkyl phosphonic acids, or alkyl phosphinic acids, however, other coordinating agents, such as pyridines, furans, and amines may also be suitable for the nanocrystal production. Examples of suitable coordinating agents include pyridine, tri-n-octyl phosphine (TOP) and tri-n-octyl phosphine oxide (TOPO). Technical grade TOPO can be used.

The outer surface of the nanocrystal can include a layer of compounds derived from the coordinating agent used during the growth process. The surface can be modified by repeated exposure to an excess of a competing coordinating group to form an overlayer. For example, a dispersion of the capped nanocrystal can be treated with a coordinating organic compound, such as pyridine, to produce crystallites which disperse readily in pyridine, methanol, and aromatics but no longer disperse in aliphatic solvents. Such a surface exchange process can be carried out with any compound capable of coordinating to or bonding with the outer surface of the nanocrystal, including, for example, phosphines, thiols, amines and phosphates. The nanocrystal can be exposed to short chain polymers which exhibit an affinity for the surface and which terminate in a moiety having an affinity for a suspension or dispersion medium. Such affinity improves the stability of the suspension and discourages flocculation of the nanocrystal.

Monodentate alkyl phosphines (and phosphine oxides, the term phosphine below will refer to both) can passivate nanocrystals efficiently. When nanocrystals with conventional monodentate ligands are diluted or embedded in a non-passivating environment (i.e. one where no excess ligands are present), they tend to lose their high luminescence and their initial chemical inertness. Typical are an abrupt decay of luminescence, aggregation, and/or phase separation. In order to overcome these limitations, polydentate ligands can be used, such as a family of polydentate oligomerized phosphine ligands. The polydentate ligands show a high affinity between ligand and nanocrystal nanocrystal surface. In other words, they are stronger ligands, as is expected from the chelate effect of their polydentate characteristics. Oligomeric phosphines have more than one binding site to the nanocrystal surface, which ensures their high affinity to the nanocrystal surface. See, for example, for example, U.S. Ser. No. 10/641,292, filed Aug. 25, 2003, and U.S. Ser. No. 60/403,367, filed Aug. 15, 2002, each of which is incorporated by reference in its entirety. The oligomeric phosphine can be formed from a monomeric, polyfunctional phosphine, such as, for example, trishydroxypropylphosphine, and a polyfunctional oligomerization reagent, such as, for example, a diisocyanate. The oligomeric phosphine can be contacted with an isocyanate of formula R′-L-NCO, wherein L is C₂-C₂₄ alkylene, and R′ has the formula 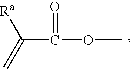
R′ has the formula 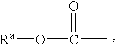
or R′ is hydrogen, wherein R^{a} is hydrogen or C₁-C₄ alkyl.

Bioconjugation to the outer surface of nanocrystals can be accomplished. For example, nanocrystals with oligomeric phosphine with carboxylic acid can be coupled to amine-derivatized biomolecules via carbodiimide couplings using EDC(1-ethyl-3-(3-dimethylaminopropyl)carbodiimide hydrochloride). The general coupling reaction is described, for example, in Hermanson, G. T. Bioconjugate Techniques 1996 Academic Press, which is incorporated by reference in its entirety. Elecrostatic interactions can be also used as thiol-based ligands with carboxylic acid. See, for example, Mattoussi, H., et al., J. Am. Chem. Soc. 2000, 122, 12142, and Goldman, E. R., et al., 2002 J. Am. Chem. Soc 124, 6378, each of which is incorporated by reference in its entirety. Nanocrystals with small oligomeric phosphine can be coupled to many biomolecules using carbonyldiimidazole or epichlorohydrin. See, for example, Pathak S., et al., 2001 J. Am. Chem. Soc 123, 4103, and Hermanson, G. T. Bioconjugate Techniques 1996 Academic Press, each of which is incorporated by reference in its entirety.

The nanocrystal can be a member of a population of nanocrystals having a narrow size distribution. The nanocrystal can be a sphere, rod, disk, or other shape. The nanocrystal can include a core of a semiconductor material. The nanocrystal can include a core having the formula MX, where M is cadmium, zinc, magnesium, mercury, aluminum, gallium, indium, thallium, or mixtures thereof, and X is oxygen, sulfur, selenium, tellurium, nitrogen, phosphorus, arsenic, antimony, or mixtures thereof.

The semiconductor forming the core of the nanocrystal can include Group II-VI compounds, Group II-V compounds, Group III-VI compounds, Group III-V compounds, Group IV-VI compounds, Group I-III-VI compounds, Group II-IV-VI compounds, and Group II-IV-V compounds, for example, ZnS, ZnSe, ZnTe, CdS, CdSe, CdTe, HgS, HgSe, HgTe, AlN, AlP, AlAs, AlSb, GaN, GaP, GaAs, GaSb, GaSe, InN, InP, InAs, InSb, TlN, TlP, TIAs, TlSb, PbS, PbSe, PbTe, or mixtures thereof.

The quantum efficiency of emission from nanocrystals having a core of a first semiconductor material be enhanced by applying an overcoating of a second semiconductor material such that the conduction band of the second semiconductor material is of higher energy than that of the first semiconductor material, and the valence band of the second semiconductor material is of lower energy than that of the first semiconductor material. As a result, carriers, i.e., electrons and holes, are confined in the core of the nanocrystal. The core can have an overcoating on a surface of the core. The overcoating can be a semiconductor material having a composition different from the composition of the core, and can have a band gap greater than the band gap of the core. The overcoat of a semiconductor material on a surface of the nanocrystal can include a Group II-VI compounds, Group II-V compounds, Group III-VI compounds, Group III-V compounds, Group IV-VI compounds, Group I-III-VI compounds, Group II-IV-VI compounds, and Group II-IV-V compounds, for example, ZnS, ZnSe, ZnTe, CdS, CdSe, CdTe, HgS, HgSe, HgTe, AlN, AlP, AlAs, AlSb, GaN, GaP, GaAs, GaSb, GaSe, InN, InP, InAs, InSb, TlN, TlP, TlAs, TlSb, PbS, PbSe, PbTe, or mixtures thereof.

The emission from the nanocrystal can be a narrow Gaussian emission band that can be tuned through the complete wavelength range of the ultraviolet, visible, or infrared regions of the spectrum by varying the size of the nanocrystal, the composition of the nanocrystal, or both. For example, CdSe can be tuned in the visible region and InAs can be tuned in the infrared region.

The population of nanocrystals can have a narrow size distribution. The population can be monodisperse and can exhibit less than a 15% rms deviation in diameter of the nanocrystals, preferably less than 10%, more preferably less than 5%. Spectral emissions in a narrow range of between 10 and 100 nm full width at half max (FWHM) can be observed. Semiconductor nanocrystals can have emission quantum efficiencies of greater than 2%, 5%, 10%, 20%, 40%, 60%, 70%, or 80%.

Methods of preparing semiconductor nanocrystals include pyrolysis of organometallic reagents, such as dimethyl cadmium, injected into a hot, coordinating agent. This permits discrete nucleation and results in the controlled growth of macroscopic quantities of nanocrystals. Preparation and manipulation of nanocrystals are described, for example, in U.S. application Ser. No. 08/969,302, incorporated herein by reference in its entirety. The method of manufacturing a nanocrystal is a colloidal growth process and can produce a monodisperse particle population. Colloidal growth occurs by rapidly injecting an M donor and an X donor into a hot coordinating agent. The injection produces a nucleus that can be grown in a controlled manner to form a nanocrystal. The reaction mixture can be gently heated to grow and anneal the nanocrystal. Both the average size and the size distribution of the nanocrystals in a sample are dependent on the growth temperature. The growth temperature necessary to maintain steady growth increases with increasing average crystal size. The nanocrystal is a member of a population of nanocrystals. As a result of the discrete nucleation and controlled growth, the population of nanocrystals obtained has a narrow, monodisperse distribution of diameters. The monodisperse distribution of diameters can also be referred to as a size. The process of controlled growth and annealing of the nanocrystals in the coordinating agent that follows nucleation can also result in uniform surface derivatization and regular core structures. As the size distribution sharpens, the temperature can be raised to maintain steady growth. By adding more M donor or X donor, the growth period can be shortened.

An overcoating process is described, for example, in U.S. application Ser. No. 08/969,302, incorporated herein by reference in its entirety. By adjusting the temperature of the reaction mixture during overcoating and monitoring the absorption spectrum of the core, over coated materials having high emission quantum efficiencies and narrow size distributions can be obtained. Alternatively, an overcoating can be formed by exposing a core nanocrystal having a first composition and first average diameter to a population of nanocrystals having a second composition and a second average diameter smaller than the first average diameter.

The M donor can be an inorganic compound, an organometallic compound, or elemental metal. M is cadmium, zinc, magnesium, mercury, aluminum, gallium, indium or thallium. The X donor is a compound capable of reacting with the M donor to form a material with the general formula MX. Typically, the X donor is a chalcogenide donor or a pnictide donor, such as a phosphine chalcogenide, a bis(silyl) chalcogenide, dioxygen, an ammonium salt, or a tris(silyl) pnictide. Suitable X donors include dioxygen, bis(trimethylsilyl) selenide ((TMS)₂Se), trialkyl phosphine selenides such as (tri-n-octylphosphine) selenide (TOPSe) or (tri-n-butylphosphine) selenide (TBPSe), trialkyl phosphine tellurides such as (tri-n-octylphosphine) telluride (TOPTe) or hexapropylphosphorustriamide telluride (HPPTTe), bis(trimethylsilyl)telluride ((TMS)₂Te), bis(trimethylsilyl)sulfide ((TMS)₂S), a trialkyl phosphine sulfide such as (tri-n-octylphosphine) sulfide (TOPS), an ammonium salt such as an ammonium halide (e.g., NH₄Cl), tris(trimethylsilyl) phosphide ((TMS)₃P), tris(trimethylsilyl) arsenide ((TMS)₃As), or tris(trimethylsilyl) antimonide ((TMS)₃Sb). In certain embodiments, the M donor and the X donor can be moieties within the same molecule.

The semiconductor nanocrystal can emit light in the near infrared (NIR) or infrared (IR) wavelength regions when excited with incident radiation. An example of a semiconductor nanocrystal that emits light in the near infrared or infrared wavelength regions is a semiconductor nanocrystal heterostructure, which has a core of a first semiconductor material surrounded by an overcoating of a second semiconductor material. The first semiconductor material and second semiconductor material are selected so that, upon excitation, one carrier is substantially confined to the core and the other carrier is substantially confined to the overcoating. See, for example, U.S. Ser. No. 10/638,546, filed Aug. 12, 2003, and U.S. Ser. No. 60/402,726, filed Aug. 13, 2002, which is incorporated by reference in its entirety.

In one example, the conduction band of the first semiconductor material is at higher energy than the conduction band of the second semiconductor material and the valence band of the first semiconductor material is at higher energy than the valence band of the second semiconductor material. In another example, the conduction band of the first semiconductor material is at lower energy than the conduction band of the second semiconductor material and the valence band of the first semiconductor material is at lower energy than the valence band of the second semiconductor material. These band alignments make spatial separation of the hole and the electron energetically favorable upon excitation. These structures are type II heterostructures. In contrast, the configurations in which the conduction band of the second semiconductor material is of higher energy than that of the first semiconductor material, and the valence band of the second semiconductor material is of lower energy than that of the first semiconductor material are type I heterostructures. The language of type I and type II is borrowed from the quantum well literature where such structures have been extensively studied.

Nanocrystals having type II heterostructures have advantageous properties that result of the spatial separation of carriers. In some nanocrystals having type II heterostructures the effective band gap, as measured by the difference in the energy of emission and energy of the lowest absorption features, can be to the red of either of the two semiconductors making up the structure. By selecting particular first semiconductor materials and second semiconductor materials, and core diameters and overcoating thicknesses, nanocrystals having type II heterostructures can have emission wavelengths previously unavailable with the semiconductor of the nanocrystal core in previous structures. In addition, the separation of charges in the lowest excited states of nanocrystals having type II heterostructures can make these materials more efficient in photovoltaic or photoconduction devices where the nanocrystals are chromophores and one of the carriers needs to be transported away from the excitation site prior to recombination.

Advantageously, a wide variety of nanocrystals having type II heterostructures can be prepared using colloidal synthesis. Colloidal synthesis allows nanocrystals to be prepared with controllable dispersibility imparted from coordinating agents, such as ligands, and are prepared in the absence of wetting layers commonly employed in nanocrystals having type II heterostructures prepared by molecular beam epitaxy.

The overcoating can be a semiconductor material having a composition different from the composition of the core which is selected to provide a type II heterostructure. The overcoat of a semiconductor material on a surface of the nanocrystal can include a Group II-VI compounds, Group II-V compounds, Group III-VI compounds, Group Ill-V compounds, Group IV-VI compounds, Group I-III-VI compounds, Group II-IV-VI compounds, and Group II-IV-V compounds, for example, ZnS, ZnSe, ZnTe, CdS, CdSe, CdTe, HgS, HgSe, HgTe, AIN, AIP, AlAs, AlSb, GaN, GaP, GaAs, GaSb, GaSe, InN, InP, InAs, InSb, TIN, TIP, TlAs, TlSb, PbS, PbSe, PbTe, or mixtures thereof. For example, ZnS, ZnSe or CdS overcoatings can be grown on CdSe or CdTe nanocrystals.

Size distribution during the growth stage of the reaction can be estimated by monitoring the absorption line widths of the particles. Modification of the reaction temperature in response to changes in the absorption spectrum of the particles allows the maintenance of a sharp particle size distribution during growth. Reactants can be added to the nucleation solution during crystal growth to grow larger crystals. By stopping growth at a particular nanocrystal average diameter, a population having an average nanocrystal diameter of less than 150 Å can be obtained. A population of nanocrystals can have an average diameter of 15 Å to 125 Å.

The particle size distribution can be further refined by size selective precipitation with a poor solvent for the nanocrystals, such as methanol/butanol as described in U.S. application Ser. No. 08/969,302, incorporated herein by reference in its entirety. For example, nanocrystals can be dispersed in a solution of 10% butanol in hexane. Methanol can be added dropwise to this stirring solution until opalescence persists. Separation of supernatant and flocculate by centrifugation produces a precipitate enriched with the largest crystallites in the sample. This procedure can be repeated until no further sharpening of the optical absorption spectrum is noted. Size-selective precipitation can be carried out in a variety of solvent/nonsolvent pairs, including pyridine/hexane and chloroform/methanol. The size-selected nanocrystal population can have no more than a 15% rms deviation from mean diameter, preferably 10% rms deviation or less, and more preferably 5% rms deviation or less.

Transmission electron microscopy (TEM) can provide information about the size, shape, and distribution of the nanocrystal population. Powder x-ray diffraction (XRD) patterns can provided the most complete information regarding the type and quality of the crystal structure of the nanocrystals. Estimates of size are also possible since particle diameter is inversely related, via the X-ray coherence length, to the peak width. For example, the diameter of the nanocrystal can be measured directly by transmission electron microscopy or estimated from x-ray diffraction data using, for example, the Scherrer equation. It also can be estimated from the UV/Vis absorption spectrum.

The nanocrystal can be incorporated into composition, such as an injectable preparation that can include an acceptable diluent, or a slow release matrix in which the nanocrystal is imbedded. The composition can be provided in a container, pack, or dispenser together with instructions for administration. The composition can be formulated in accordance with their intended route of administration. Acceptable routes include oral or parenteral routes (e.g., intravenous, intradermal, transdermal (e.g., subcutaneous or topical), or transmucosal (i.e., across a membrane that lines the respiratory or anogenital tract). The compositions can be formulated as a solution or suspension and, thus, can include a sterile diluent (e.g., water, saline solution, a fixed oil, polyethylene glycol, glycerine, propylene glycol or another synthetic solvent); an antimicrobial agent (e.g., benzyl alcohol or methyl parabens; chlorobutanol, phenol, ascorbic acid, thimerosal, and the like); an antioxidant (e.g., ascorbic acid or sodium bisulfite); a chelating agent (e.g., ethylenediaminetetraacetic acid); or a buffer (e.g., an acetate-, citrate-, or phosphate-based buffer). When necessary, the ph of the solution or suspension can be adjusted with an acid (e.g., hydrochloric acid) or a base (e.g., sodium hydroxide). Proper fluidity (which can ease passage through a needle) can be maintained by a coating such as lecithin, by maintaining the required particle size (in the case of a dispersion), or by the use of surfactants. The body can be an animal (e.g., a rabbit, mouse, guinea pig, rat, horse, cow, pig, dog, cat or human).

### EXAMPLES

Animals. Animals were used in accordance with an approved institutional protocol. Male Sprague-Dawley rats were from Charles River Laboratories (Wilmington, Mass.). Hairless athymic nu/nu mice were from Taconic (Germantown, N.Y.). Rats and mice were anesthetized with 65 mg/kg and 50 mg/kg intraperitoneal pentobarbital, respectively.

Reagents. Sterile Intralipid™ (20%) was purchased from Baxter (Deerfield, Ill.). Water was purified on a Milli-Q system (Millipore, Bedford, Mass.). Olive oil was from Filippo Berio (Viareggio, Italy). Oxyhemoglobin (OxyHb) was prepared from normal human donors as described in Drabkin, J. Biol. Chem. 164:703-723 (1946), which is incorporated by reference in its entirety. Deoxyhemoglobin (DeoxyHb) was prepared by treatment of OxyHb with 1% sodium dithionite (Sigma, St. Louis, Mo.). Albumin, Cohn Fraction V was also from Sigma. All solutions except Intralipid were filtered through 0.2 μm filters (Millipore) prior to use to eliminate scatter. Trioctylphosphine oxide (TOPO), selenium shot, and tellurium shot were from Alfa Aesar (Ward Hill, Mass.). Trioctylphosphine (TOP) was from Fluka (St. Louis, Mo.). Hexadecylamine (HDA) was from Aldrich (St. Louis, Mo.). All other reagents were purchased from Fisher Scientific (Hanover Park, Ill.).

Preparation of Aqueous Soluble 752 nm CdTe(CdSe) core(shell) semiconductor nanocrystals. The CdTe(CdSe) composition included a core of cadmium telluride (CdTe) and a thin shell of cadmium selenide (CdSe). Unless otherwise noted, all reactions were carried out in a dry nitrogen atmosphere using a glove box or standard Schlenk techniques. 1.5 M Trioctylphosphine selenide (TOPSe) and 0.5 M trioctylphosphine telluride (TOPTe) were prepared by adding selenium (or tellurium) shot to TOP and stirring until dissolved. 554 mg cadmium acetylacetonate (CdAcAc) was suspended in 6.0 ml TOP and stirred under vacuum at 100° C. until well mixed. A nitrogen atmosphere was then introduced, and the mixture was cooled to room temperature. 0.4 ml TOPSe and 2.7 ml TOPTe were then added and stirred. A mixture of 6.25 g TOPO and 5.75 g HDA was dried under vacuum at 130° C. in a round bottom flask, then filled with nitrogen and heated to 350° C. The CdAcAc, TOPSe, and TOPTe mixture was diluted by a small amount of TOP, and then injected into this flask. The reaction mixture was stirred at 250° C. until the desired particle size was reached according to the absorption spectrum of the mixture. The heat was removed, and hexane was added when the temperature reached approximately 60° C. The mixture was centrifuged and a precipitate consisting of reaction by-products was discarded. Methanol was added to precipitate the semiconductor nanocrystals. The mixture was centrifuged and decanted yielding a powder of semiconductor nanocrystals. Extensive characterization of such semiconductor nanocrystals using transmission electron microscopy (TEM), x-ray diffraction and fluorescence lifetime measurements show that the structure is consistent with the core consisting of CdTe and a shell of CdSe. See, for example, U.S. Ser. No. 10/638,546, filed Aug. 12, 2003, U.S. Ser. No. 09/732,013, filed Dec. 8, 2000, and U.S. Ser. No. 60/402,726, filed Aug. 13, 2002, each of which is incorporated by reference in its entirety.

The semiconductor nanocrystals were dispersed in water by exchanging the organic caps with oligomeric phosphines derivatized with carboxylic acid (OPCA) as follows. See, for example, U.S. Ser. No. 10/641,292, filed Aug. 15, 2003, and U.S. Ser. No. 60/403,367, filed Aug. 15, 2002, which is incorporated by reference in its entirety. The semiconductor nanocrystal sample was re-dispersed in chloroform. A water solution of OPCA was introduced, forming a bilayer. This mixture was sonicated until all the semiconductor nanocrystals were transferred to the aqueous phase, as determined by the transfer of color from the organic phase to the aqueous phase. Excess OPCA was removed by dialysis. Concentration was determined as described in Leatherdale et al., J. Phys. Chem. B. 106:7619-7622 (2002), which is incorporated by reference in its entirety.

Aqueous Solubility, Absorbance, Fluorescence Emission, and Quantum Yield: Extensive characterization using transmission electron microscopy, x-ray diffraction and fluorescence lifetime measurements show that the structure is consistent with a core consisting of CdTe and a shell of CdSe (data not shown), and a mean diameter of approximately 10 nm. Nanocrystals were made soluble in aqueous media by treatment with oligomeric phosphines as described above. The extinction coefficient of these NIR nanocrystals feature the characteristic increase to the blue, with a shoulder at approximately 730 nm. Scanning spectrofluorometry showed a peak emission at 752 nm, with a full-width half-maximum (FWHM) of 60 nm. The QY in PBS was approximately 25% using Oxazine 725 laser dye as a reference.

Nanocrystals were synthesized in organic solvents and are not usually soluble in aqueous environments. To make soluble, a coating has to be used. The particular coating used in this invention is a polydentate phosphine ligand (OPCA).

Example of a procedure for synthesizing OPCA and ligand exchange:

Synthesis of the OPCA ligand: 8.00 g Trishydroxypropylphosphine (THPP,Strem, 90%) was dissolved in 20.0 g Dimethylformamide (DMF, Aldrich, 99.8%). 4.54 g Diisocyanatohexane (DIH, Aldrich, 98%) was added dropwise at room temperature, while the solution was vigorously stirred. The solution was stirred for a day after completion of the addition. 19.35 g Ethylisocyanatoacetate (EIA, Aldrich, 95%) was added dropwise at room temperature, and kept stirred for a day. The solvent and excess EIA are removed at 100° C. in vacuo.

Ligand-exchange of CdTe/CdSe(core/shell) QDs with oligomeric phosphine ligands: A CdTe/CdSe(core/shell) nanocrystal powder free of excess TOPO or TOP was obtained by previously described nonsolvent-precipitation methods. Anhydrous methanol is used for the nonsolvent, and the powder was collected after centrifugation. 100 mg CdTe/CdSe(core/shell) nanocrystals, 3.0 g oligomeric phosphine ligands, ˜10 mL anhydrous Tetrahydrofuran (THF), and ˜2 mL N,N-dimethylformamide (DMF) were rigorously mixed together. The amounts of THF and DMF were chosen so as to make the solution homogeneous. The solution was stirred for an hour. The solvents (THF and DMF) were removed at 100° C. in vacuum. The remaining viscous mixture solution was kept at 120° C. for 3 hours, and cooled to room temperature.

Hydrolysis of the oligomeric phosphine ligand and purification:

To the sample prepared by above, 50 mL 1.0M sodium hydroxide (NaOH) aqueous solution was added. A stir bar was placed at the interface between the viscous nanocrystal solution and the NaOH solution and stirred vigorously at room temperature. The stirring was continued until the mixture was no longer phase separated and became a slightly turbid dark brown color. The solution was passed through 0.2 μM pore-sized filters and a filtered clear solution was obtained. The solvent was exchanged to a PBS buffer by continuous diafiltration using 50K nominal molecular weight limit membranes.

Tissue Preparation. Human whole blood was collected directly into a purple top (EDTA) clinical specimen tube and stored at 4° C. Where indicated, it was diluted in phosphate-buffered saline, pH 7.4 (PBS) supplemented with 5 mM EDTA (to prevent clotting). Skin was prepared by surgical excision and bathed in ice cold PBS. Specimens were used within 3 hours of collection.

Absorbance Measurements. Pairs of optically matched 1.0 cm or 0.05 cm cuvettes (Spectrocell, Oreland, Pa.) were used on a Model 5 (Varian-Cary, Palo Alto, Calif.) scanning spectrophotometer equipped with deuterium and tungsten lamps. Absorbance wavelength scans from 400 nm to 2000 nm, at a resolution of 1 nm, were performed on water (air blank), lipid (olive oil; air blank), OxyHb in PBS, DeoxyHb in PBS, and protein (albumin) in PBS (PBS blank). Five individual scans were averaged prior to calculation of the extinction coefficient at each wavelength. Measured values matched closely those described in Conway et al., Am. J. Clin. Nutr. 40:1123-1130 (1984); and Kuenstner et al., Biospectroscopy 3:225-232 (1997), which is incorporated by reference in its entirety.

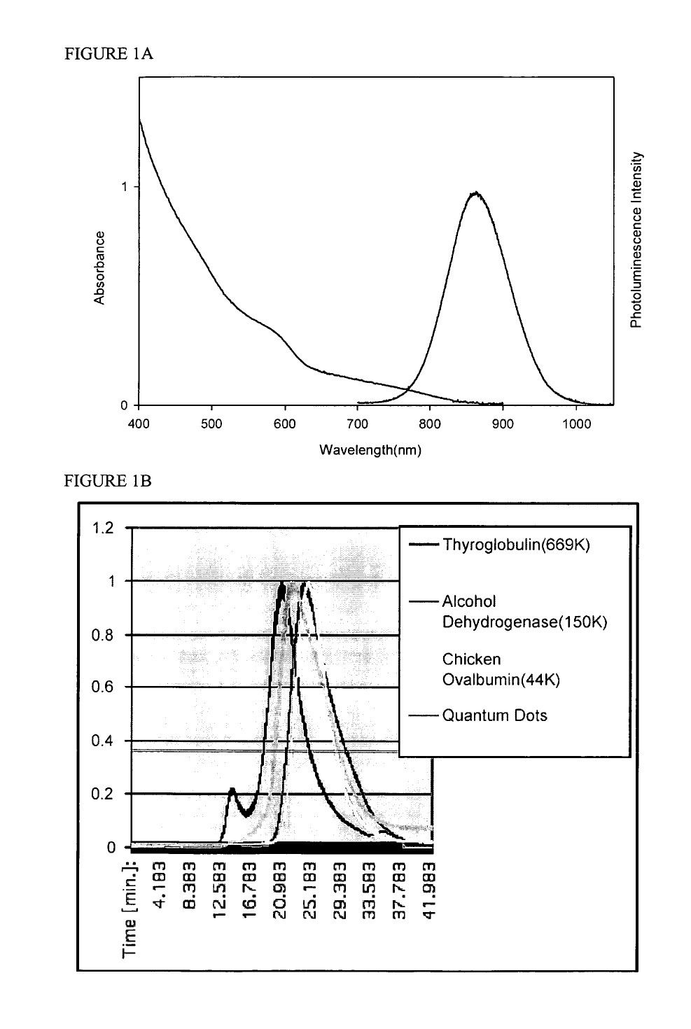

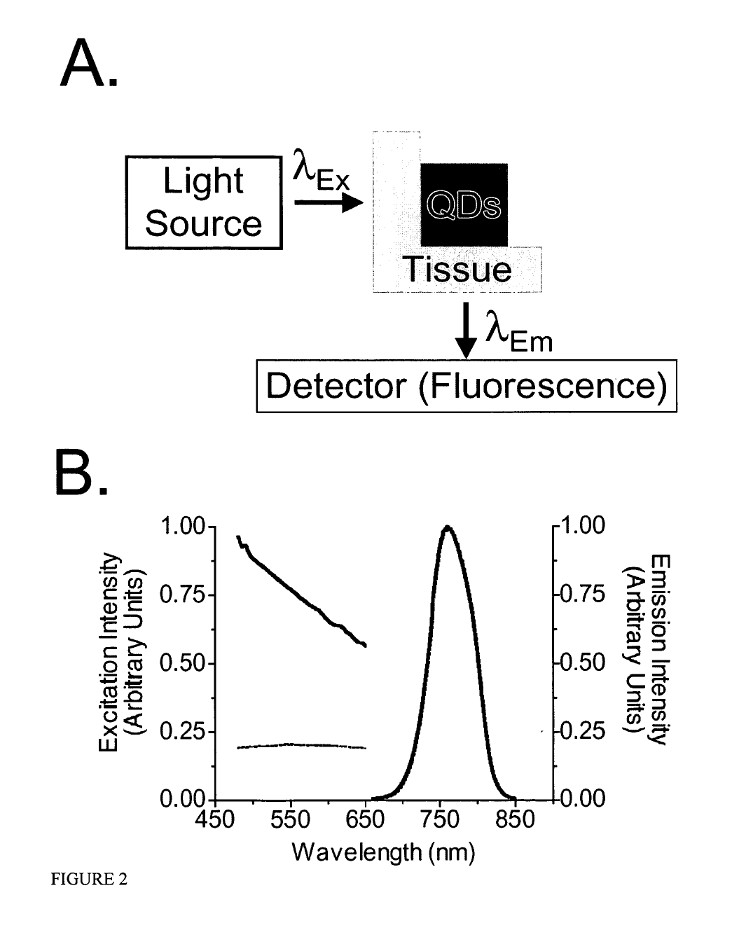

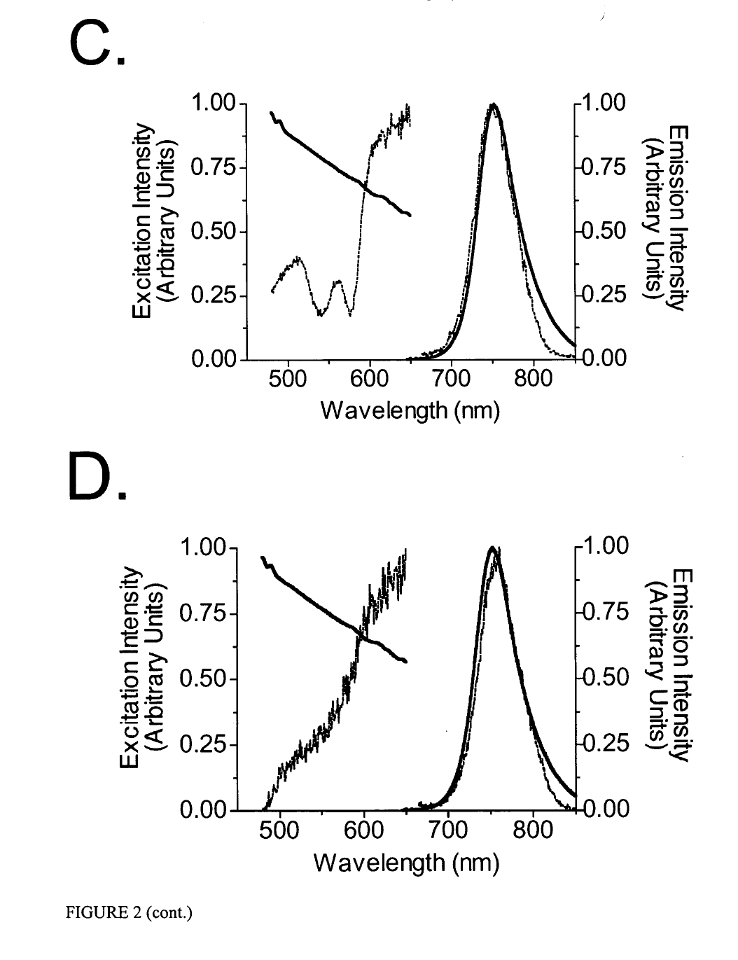

Scanning Spectrofluorometry. Fluorescence excitation and emission scans were performed on a SPEX Fluorolog-2 spectrofluorometer (Jobin Yvon Horiba, Edison, N.J.) equipped with a R928 photomultiplier tube. To preserve high quantum yield (QY), non-OPCA treated semiconductor nanocrystals were diluted to 1 μM in hexane and placed in a 1 cm path length cuvette sandwiched by different in vivo simulating media, or tissue, as shown in FIG. 2A. The semiconductor nanocrystals shown in FIG. 1 were subjected to excitation and emission spectrofluorometry using the geometry shown in FIG. 2A. Referring to FIG. 1, CdTe(CdSe) core(shell) semiconductor nanocrystals with peak fluorescent emission at 752 nm were prepared and resuspended in PBS at a concentration of 1 μM. Extinction coefficient is shown on the left axis (thick solid line) and photoluminescence (500 nm excitation) is shown on the right axis (dashed line), both as a function of wavelength. Referring to FIG. 2A, for excitation scans (left curves), the emission wavelength was fixed at 752 nm. For emission scans (right curves), the excitation wavelength was fixed at 550 nm (Intralipid) or 650 nm (blood, skin). The illumination/detection geometry of spectrophotometer experiments is shown. Excitation light (λ_{Ex}) was a single, thin collimated beam propagating through optically thin tissue. Semiconductor nanocrystals at the given concentration are below the tissue and absorb the net excitation photons. Depending on the quantum yield of the semiconductor nanocrystals, fluorescent light (λ_{Em}) was emitted and propagates out through the same thickness of tissue. The detector was placed at 90° relative to the excitation light beam. For comparison, semiconductor nanocrystal performance in PBS is shown on each graph (thick solid line). Data are normalized for display on a single ordinate.

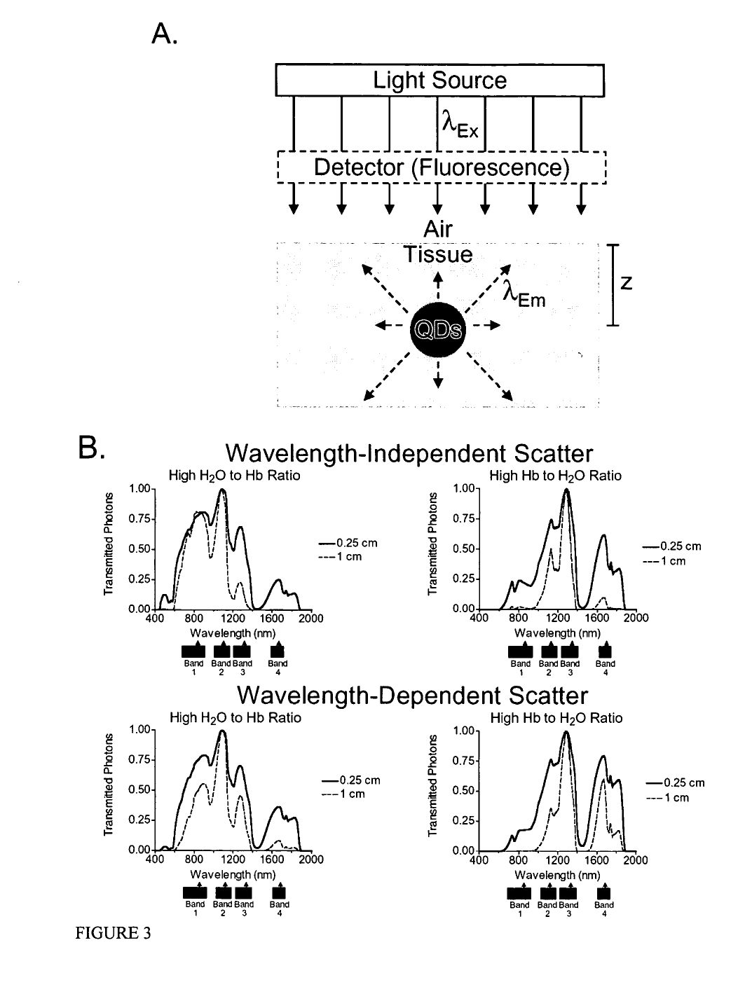

Modeling semiconductor nanocrystal Performance during In Vivo Imaging. To describe light propagation through tissue, the geometry shown in FIG. 3A was assumed and adapted a previously described analytical solution to the diffusion equation. See, for example, Gardner et al., Lasers Surg. Med. 18:129-38 (1996), which is incorporated by reference in its entirety. Briefly, for a given fluence rate, the local rate of energy absorption by semiconductor nanocrystals (R_{A} in mW/cm³) can be expressed by the extinction (or absorption) coefficient of semiconductor nanocrystals at the excitation wavelength (λ_{Ex}) as (μ_{QDs}(λ_{Ex})(cm⁻¹)) and the spatial distribution of the light energy fluence rate φ(z, λ_{Ex}), in mW/cm², where z represents depth in the tissue:
R_{A}(z, λ_{Ex})=μ_{QDs}(λ_{Ex})●φ(z, λ_{Ex})

where μ_{QDs}(λ_{Ex})=ε_{QDs}(λ_{Ex})●c_{semiconductor nanocrystals}, ε_{QDs}(λ_{Ex}) is the extinction coefficient per mole of semiconductor nanoc cystals and c_{semiconductor nanocrystals} is the molar concentration of semiconductor nanocrystals. Since c_{semiconductor nanocrystals} did not affect any of the results discussed below, it was held constant in all simulations. The fluence rate φ(z, λ_{Ex}) is given by:
φ(z, λ_{Ex})=E_{O}[D₁exp(−K₁z/δ)−D₂exp(−k₂z/δ)]

where Eₒ (mW/cm²) is the incident fluence rate (for all simulations, E₀ was held constant at 50 mW/cm² at each wavelength), and δ is the effective penetration depth, defined from diffusion theory as:

$$δ = \frac{1}{\sqrt{3 {μ}_{a} ( {μ}_{a} + {μ}_{s}^{′} )}}$$

$$where {μ}_{a} = {\sum }_{i = 1} {μ ( λ )}_{a , i} {c}_{i} and {μ}_{s}^{′} = {\sum }_{i = 1} {{μ}^{′} ( λ )}_{s , i} {c}_{i}$$

Here, μₐ(λ)(cm⁻¹) and μ!ₛ(λ)(cm⁻¹)represent the total tissue absorption and reduced scattering coefficients, respectively, and μ_{a,i}(λ) (M⁻¹cm⁻¹) and μ′_{s,i}(λ)(M⁻¹cm⁻¹) represent the absorption and scatter coefficients, respectively, of individual biomolecules at the particular excitation or emission wavelength, and at a concentration cᵢ (M), which comprise the tissue. Values for μₐ of water, lipid, DeoxyHb, OxyHb. and protein were measured as described above. The relationship between scattering coefficient and wavelength (λ) can be empirically described as follows: μ′ₛ(λ)=Jλ^{-P}, where J is related to the scattering density and P is the scatter power coefficient. See, for example, Mourant et al., Appl. Opt. 36:949-957 (1997), which is incorporated by reference in its entirety. The parameters D₁, k₁, D₂, k₂ (and D₃, k₃, see below) depend solely upon diffuse reflectance, R_{d}, aspects of which have been previously investigated through Monte Carlo simulations (see, Gardner et al., Lasers Surg. Med. 18:129-138 (1996)):
D₁=3.04+4.90R_{d}−2.06exp(−21.1R_{d})
k₁=1−(1−1/√3)exp(−18.9R_{d})
D₂=2.04−1.33R_{d}−2.04exp(−21.1R_{d})
k₂=1.59exp(3.36R_{d})

For simplicity, the refractive index of tissue was assumed to be 1.33 as for all simulations. The value of R_{d} depends on the absorption coefficient of the tissue and the effective path length that photons travel in the tissue, and can be approximated as a function of N′, defined as the ratio of reduced scattering coefficient to absorption (μ′ₛ/μₐ). The diffuse reflectance, R_{d}, from the surface of a semi-infinite medium is approximated by the expression (see, Jacques, Vol. 1999, Oregon Medical Laser Center News (1999), which is incorporated by reference in its entirety):

$${R}_{d} \approx exp ( - A δ {μ}_{a} ) = exp ( - \frac{A}{\sqrt{3 ( 1 + {N}^{′} )}} )$$

$$where A = 6.3744 + 0.35688 exp ( ln ( {N}^{′} ) / 3.4739 )$$

The factor Aδ equals the apparent path length L for photon attenuation due to the absorption coefficient. A is approximately 7-8 for most soft tissues. See, Jacques, Vol. 1999, Oregon Medical Laser Center News (1999). These analytical expressions have accuracy comparable to Monte Carlo simulations over an essentially unrestricted range of diffuse reflectance values. See Gardner et al., Lasers Surg. Med. 18:129-38 (1996). The rate of semiconductor nanocrystal emission (R_{E} in mW/cm³) is given by:
R_{E}(z, λ_{Ex}, λ_{Em})=R_{A}(z, λ_{Ex})●QY(λ_{Em})●G(z, λ_{Em})

Where QY(λ_{Em}) represents the QY of semiconductor nanocrystals at the emission wavelength (λ_{Em}) G(z, λ_{Em}) or the escape function, which describes the exponential decay of emitted light from an isotropic point source at depth z (see, Gardner et al., Lasers Surg. Med. 18:129-38 (1996)), is given by:
G(z, λ_{Em})=D₃exp(−k₃z/δ)
where:
D₃=0.32+0.72R_{d}−0.16exp(−9.11R_{d})
k₃=1−0.30exp(−6.12R_{d})

In the case of broadband excitation light, the source and excitation spectrum must be integrated over all incident wavelengths. Thus, the above equation can be re-written as follows:
R_{E}(z, λ_{Ex}, λ_{Em})=Σᵢ[R_{A}(z, λ_{Ex,i})QY(λ_{Em})●G(z, λ_{Em})]

The light intensity of R_{A} or R_{E} at any one wavelength can be converted to number of photons per cm³(N_{A,E}) by the following formula:

$${N}_{A , E} = \frac{{R}_{A , E}}{1.99 \times {10}^{- 16} / λ ( nm )}$$

If desired, the geometry of the semiconductor nanocrystal source can be used to convert N_{A,E} into units of mW/cm². These equations, along with the attenuation curves for water, lipid, OxyHb, DeoxyHb, and protein were incorporated into an Excel 98 spreadsheet (Microsoft, Redmond, Wash.) for rapid analysis of model variables. The model is available from the authors as an Excel spreadsheet.

In Vivo NIR Fluorescence Imaging of the Coronary Vasculature. Anesthetized 350 g rats were ventilated on a SAR-830AP (CWE, Ardmore Pa.) ventilator and a midline sternotomy was performed. The exposed heart was imaged as described in Nakayama et al., “Functional near-infrared fluorescence imaging for cardiac surgery and targeted gene therapy,” Molecular Imaging (2002), except no laser was used, and only a single 150 W halogen light source illuminated the surgical field. A combination of hot mirrors and band pass filters (Chroma, Brattleboro, Vt.) produced broadband excitation light of 400 nm to 700 nm at a total fluence rate of 2.0 mW/cm². A 740 dcxr dichroic mirror (740 nm center point) and model D770/50 emission filter (745 nm to 795 nm) were also purchased from Chroma. The Orca-ER (Hamamatsu, Bridgewater, N.J.) NIR camera settings included gain 7 (out of 9), 2×2 binning, 640×480 pixel field of view, and exposure time of 25 msec. Color video camera (HV-D27, Hitachi, Tarrytown, N.Y.) images were acquired at 30 frames per second at a resolution of 640×480 pixels. Data were acquired and quantitated on a Macintosh computer equipped with a Digi-16 Snapper (DataCell, North Billerica, Mass.) frame grabber (for Orca-ER), CG-7 (Scion, Frederick, Md.) frame grabber (for HV-D27) and IPLab software (Scanalytics, Fairfax, Va.). Aqueous soluble 752 nm semiconductor nanocrystals were resuspended in PBS at a concentration of 2.5 μM. One ml (2.5 nmol) of this suspension was injected intravenously via tail vein and the coronary vasculature imaged as described in the text and in Nakayama et al., “Functional near-infrared fluorescence imaging for cardiac surgery and targeted gene therapy,” Molecular Imaging (2002).

Synthesis of Aqueous Soluble NIR Emitting Semiconductor Nanocrystals

Based on an analysis of transmission bands in biological tissue having different properties (discussed in detail below), NIR semiconductor nanocrystals with a peak emission wavelength at 752 nm were synthesized. Extensive characterization using transmission electron microscopy, x-ray diffraction and fluorescence lifetime measurements show that the structure is consistent with a core consisting of CdTe and a shell of CdSe (data not shown), and a mean diameter of approximately 10 nm. Semiconductor nanocrystals were made soluble in aqueous media by treatment with oligomeric phosphines. The extinction coefficient of these NIR semiconductor nanocrystals feature the characteristic increase to the blue, with a shoulder at approximately 730 nm (FIG. 1). Scanning spectrofluorometry showed a peak emission at 752 nm, with a full-width half-maximum (FWHM) of 60 nm (FIG. 1). The QY in PBS was approximately 25% using Oxazine 725 laser dye as a reference. See, for example, Sens and Drexhage, J. Lumin. 24-25:709-712 (1981), which is incorporated by reference in its entirety.

Semiconductor Nanocrystal Performance with Scattering Medium

The influence and attenuation properties of surrounding tissue on absorbance and emission properties of semiconductor nanocrystals was determined. The experimental geometry is shown in FIG. 2A. The first medium chosen was simply a non-absorbing buffer (PBS) into which was added increasing concentrations of Intralipid. Intralipid is a suspension of various lipids in water that is often used to simulate tissue scatter, and exhibits scatter that is strongly dependent on wavelength (proportional to≈λ^{-2.4}, see, for example, van Staveren et al., Applied Optics 30:4507-4514 (1991), which is incorporated by reference in its entirety). Shown in FIG. 2B ((semiconductor nanocrystal fluorescence excitation (left) and emission (right) in 0.02% Intralipid (dashed line))) is the effect of increasing scatter on NIR semiconductor nanocrystal excitation. In the absence of scatter (thick solid line), scanning spectrofluorometry confirms that fluorescence excitation matches the pattern of absorbance shown in FIG. 1. However, with as little as 0.02% Intralipid (μₛ′≈0.3 cm⁻¹ at 630 nm), increased semiconductor nanocrystal absorbance at bluer wavelengths is lost. The effect of 0.02% Intralipid on semiconductor nanocrystal emission was insignificant (FIG. 2B).

Semiconductor Nanocrystal Performance with Tissues Having Absorbance and Either Wavelength-Independent or Wavelength-Dependent Scatter

The effect of surrounding biological tissue on performance of semiconductor nanocrystals was studied. For these experiments, human whole blood was chosen as an absorbing tissue whose scatter was independent of wavelength, and non-pigmented hairless mouse skin as a tissue whose scatter was dependent on wavelength. See, for example, Cheong et al., IEEE J. Quantum Electronics 26, 2166-2195 (1990). As shown in FIG. 2C (left curves) (semiconductor nanocrystal fluorescence excitation (left) and emission (right) in human whole blood (i.e., a tissue exhibiting wavelength-independent scatter) diluted 50-fold (dashed line)), semiconductor nanocrystals surrounded by even dilute human blood had a complex wavelength-dependent excitation spectrum, which differed markedly from the predicted semiconductor nanocrystal absorbance in non-absorbing and non-scattering medium. Most importantly, increasing absorption at bluer wavelengths was absent. The wavelength dependence of emission was fairly symmetrical about the predicted peak emission of 752 nm (FIG. 2C, right curves). The loss of bluer wavelength in the semiconductor nanocrystal excitation spectrum was even more pronounced at higher blood concentrations (data not shown). As shown in FIG. 2D (left curves) (semiconductor nanocrystal fluorescence excitation (left) and emission (right) in 0.99 mm thick non-pigmented hairless mouse skin (i.e., a tissue exhibiting wavelength-dependent scatter), the excitation spectrum of semiconductor nanocrystals surrounded by hairless mouse skin exhibited essentially a compete loss of bluer wavelengths, and semiconductor nanocrystal emission was slightly red-shifted.

These data and additional simulations (not shown) indicate that biological tissue exhibits a “filter” effect that can counteract the advantageous increase in semiconductor nanocrystal absorbance at bluer wavelengths. This effect is highly dependent on the shape of the semiconductor nanocrystal absorbance curve and the shapes and strengths of the tissue absorbance and scatter attenuation curves. Furthermore, when the tissue scatter power coefficient is high, there can be a red shift of peak semiconductor nanocrystal emission.

Selection of Semiconductor Nanocrystal Peak Emission Wavelengths Based on Tissue Transmission Bands

For reflectance fluorescence imaging, the light source is typically uniform and diffuse, and perpendicular to the air/tissue interface. Since semiconductor nanocrystals can be used for tumor targeting, and specifically for the detection of small collections of malignant cells, in the analysis they are assumed to be concentrated at a point, at a depth z below the air/tissue interface (FIG. 3A). The illumination/detection geometry used for predicting the performance of semiconductor nanocrystals for reflectance fluorescence imaging assumes continuous wave, uniform irradiance normal to the air/tissue interface, a semi-infinite thick tissue, and a point of semiconductor nanocrystals embedded in the tissue at a given depth. Fluorescent light emitted by the semiconductor nanocrystals propagates back through the tissue and is detected at 0° relative to excitation light. Adapted from Gardner et al., Lasers Surg. Med. 18:129-138 (1996). An analytical solution to the diffusion equation that matches this imaging geometry, and have validated its accuracy against Monte Carlo simulations is described in, for example, Gardner et al., Lasers Surg. Med. 18:129-38 (1996).

Using this model in spreadsheet format, semiconductor nanocrystal performance can be simulated under conditions of varying absorbance, scatter, tissue thickness, and semiconductor nanocrystal optical properties. In most tissues, absorbance is dominated by H₂O and hemoglobin (Hb), each of which has local minima and maxima of transmission. Referring to FIG. 3B, using the model geometry shown in FIG. 3A, the number of transmitted photons as a function of wavelength was simulated on tissues of high H₂O to Hb ratio (left panels) or high Hb to H₂O ratio (right panels), at tissue thicknesses of 0.25 cm (thick solid line) or 1 cm (dashed line). Simulated tissues exhibited either wavelength-independent scatter (upper panels) or wavelength-dependent scatter (lower panels). The analysis identified four possible transmission bands (black bars below ordinate) as described in the text. The arrow above each transmission band identifies the peak semiconductor nanocrystal emission wavelength used for subsequent analysis. Although total photon transmission is a continuum, to simplify the analysis, four transmission “bands” shown in FIG. 3B were studied: 690 nm to 915 nm (Band 1), 1025 to 1150 (Band 2), 1225 nm to 1370 nm (Band 3), and 1610 nm to 1710 nm (Band 4). The lower limit of Band 1 is bounded by Hb absorbance, whereas its upper limit is bounded by lipid and H₂O absorbance. All other transmission bands represent local minima in the H₂O absorption curve. Band 4 ends at 1710 in this analysis to avoid a sharp lipid absorbance peak at 1735 nm (data not shown). See, for example, Kou, L. et al., Appl. Opt. 32:3531-3540 (1993), which is incorporated by reference in its entirety.

Shown simulated in FIG. 3B are the number of photons transmitted through tissues of varying H₂O to Hb ratio, scatter power coefficient, and thickness, as a function of wavelength. For simplicity, the OxyHb to DeoxyHb ratio was fixed at one to one, and lipid content was fixed at 15% by weight (i.e., 0.25 M assuming an average lipid molecular weight of 600 Da and an equal ratio of cholesterol and phosphatidylcholine). Model parameters used for the simulation included: thickness as shown, water content=75%, lipid content=0.25 M, OxyHb concentration=1.25 or 0.02 mM, DeoxyHb concentration=1.25 or 0.02 mM, protein concentration=2.5 mM, absolute scatter at 630 nm=8.9 cm⁻¹ (wavelength-independent scatter) or 23 cm⁻¹ (wavelength-dependent scatter), and scatter power coefficient=0 (wavelength-independent scatter) or 2.81 (wavelength-dependent scatter). These model parameters were chosen to match previously described parameters (see, Cheong et al., IEEE J. Quantum Electronics 26:2166-2195 (1990)) for blood (wavelength-independent scatter) and skin (wavelength-dependent scatter). 400 nm was chosen as a lower limit for the simulation since ultraviolet light penetrates poorly into tissue, and 2000 nm was chosen as the upper limit due to water's extreme absorption above this wavelength.

For a scatter power coefficient of zero (i.e., wavelength-independent scatter; FIG. 3B, upper), relative transmission was highly influenced by both the H₂O to Hb ratio and tissue thickness. In particular, at a high Hb to H2O ratio, transmission through Bands 1,2 and 4 decreased more rapidly than through Band 3 with increasing thickness, and at all H2O to Hb ratios, transmission through Band 4 had the most rapid decrease with increasing thickness. Relative transmission through Bands 1 and 2 were affected similarly by tissue thickness (significantly less than through Bands 3 and 4) in the presence of a high H2O to Hb ratio, and transmission through Band 3 was the least affected by tissue thickness in the presence of a high Hb to H₂O ratio.

For a high scatter power coefficient (i.e., wavelength-dependent scatter; FIG. 3B, lower) the patterns of transmission were similar to those found for wavelength-independent scatter, but overall, relative transmission favored longer wavelengths. These transmission results are consistent with previous empirical measurements and serve to guide the choice of optimal semiconductor nanocrystal emission wavelengths. See, for example, Wan et al., Photochem. Photobiol. 34:679-681 (1981); Anderson and Parrish, J. Invest. Dermatol. 77:13-19 (1981); and Du et al., Phys. Med. Biol. 46:167-81 (2001), which is incorporated by reference in its entirety.

Simulated Performance of Various NIR and IR Semiconductor Nanocrystal Contrast Agents

The performance of semiconductor nanocrystals with peak emission in Bands 1 through 4 after embedding in tissues with varying H₂O to Hb ratios and scatter power coefficients was simulated. For simplicity, tissue thickness was fixed at 0.5 cm. Semiconductor nanocrystal peak emission was chosen at two thirds the width of the transmission band to provide enough bandwidth to accommodate excitation close to the emission wavelength (if needed) and the broader emission curves associated with longer wavelength semiconductor nanocrystals. To eliminate variability due to the shape of the semiconductor nanocrystal absorption curve (itself a function of the semiconductor materials used and particular preparation; see Discussion) and aqueous QY (a function of the semiconductor materials and surface coating), these parameters were fixed. In particular, the shape of the semiconductor nanocrystal absorption curve used in the simulation is common to many different types of semiconductor nanocrystal materials. See, for example, Leatherdale et al., J. Phys. Chem. B. 106:7619-7622 (2002); Guzelian et al., Applied Physics Letters 69, 1432-1434 (1996); Cao and Banin, J. Am. Chem. Soc. 122:9692-9702 (2000); and Murray et al., IBM Journal of Research and Development 45:47-56 (2001), each of which is incorporated by reference in its entirety. The emission curve was simulated with a Gaussian distribution. FWHMs for 840 nm, 1110 nm, 1320 nm, and 1680 nm semiconductor nanocrystals were chosen based on literature and empirical data, and were 76 mn, 104 mn, 145 nm, and 235 mn, respectively. QY was fixed at 50%, and the extinction coefficient at the first absorption peak was fixed at 1×10⁶ M⁻¹cm⁻¹. Other model parameters used for this simulation included: broadband excitation from 400 nm to the peak emission wavelength using a constant fluence rate at each wavelength, thickness 0.5 cm, water content=75%, lipid content=0.25 M, OxyHb concentration=1.25 or 0.02 mM (1 to 1 ratio with DeoxyHb), DeoxyHb concentration=1.25 or 0.02 mM, protein concentration=2.5 mM, absolute scatter at 630 nm=8.9 cm⁻¹ (wavelength-independent scatter) or 23 cm⁻¹ (wavelength-dependent scatter), and scatter power coefficient=0 (wavelength-independent scatter) or 2.81 (wavelength-dependent scatter).

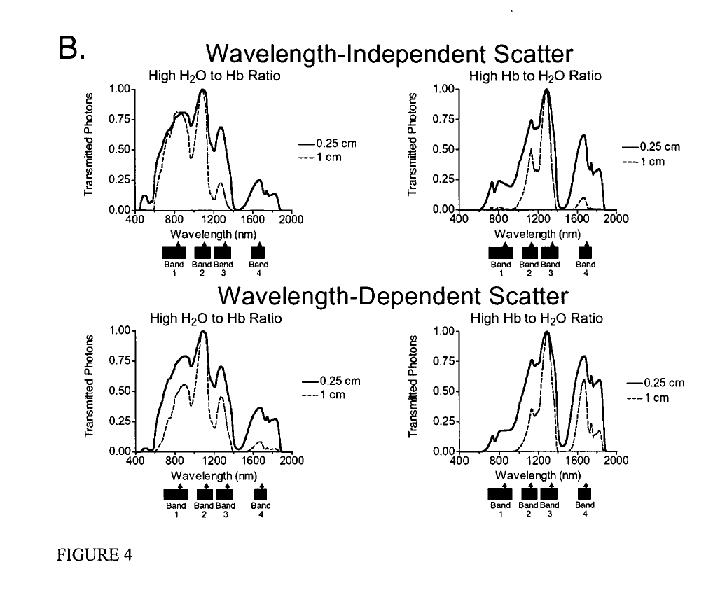

As shown in FIG. 4, semiconductor nanocrystal performance was predicted to be affected significantly by tissue optical properties. Absorbance scans for various semiconductor nanocrystals embedded in tissue with either wavelength-independent scatter (left panels) or wavelength-dependent scatter (right panels) were simulated as described in the text. Simulations were run in the presence of PBS only (thick solid line) or the presence of 0.5 cm of tissue with a high H₂O to Hb ratio (thin solid line) or high Hb to H₂O ratio (dashed line) as described in the text. Semiconductor nanocrystal peak emission is shown along the left edge of the page. Data are normalized for display on a single ordinate. Specifically, for tissues with a high H₂O to Hb ratio (thin solid curves), the key feature of semiconductor nanocrystal excitation at bluer wavelengths was often preserved, suggesting that broadband excitation light can be used. However, at a high Hb to H₂O ratio (dashed curves), semiconductor nanocrystal excitation fell rapidly below 700 nm. When wavelength-dependent scatter was also present, excitation was further confined to a narrow band close to semiconductor nanocrystal peak emission, with high similarity to the pattern of excitation typically seen using conventional fluorophores. Semiconductor nanocrystal emission (data not shown) was also affected significantly by tissue absorbance and scatter. Specifically, a red shift in peak emission wavelength was often seen in the presence of wavelength-dependent scatter (see also FIG. 2D), and the emission of 1680 nm semiconductor nanocrystals was additionally affected by lipid absorption (not shown).

Selection of Semiconductor Nanocrystal Excitation and Emission Wavelengths Based on Photon Yield

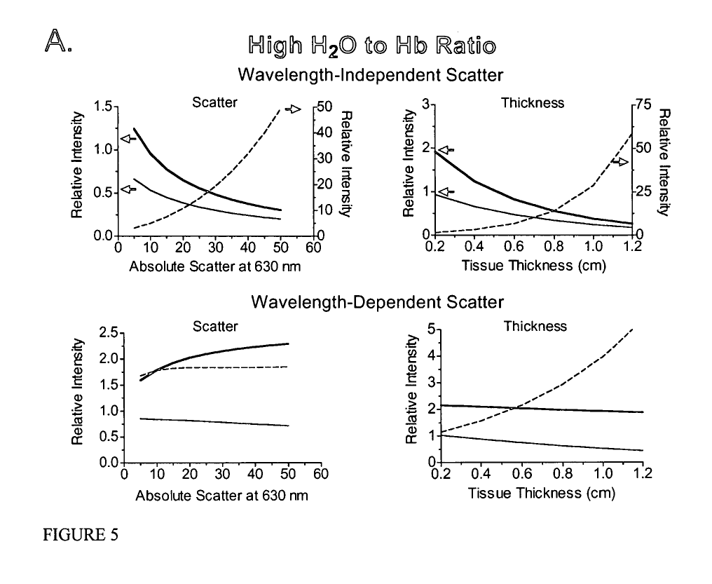

A direct comparison of semiconductor nanocrystals with emission centered at 840 nm, 1110 nm, 1320 nm, and 1680 nm, as a function of absolute scatter and tissue thickness, is presented in FIG. 5A for a high H₂O to Hb ratio, and FIG. 5B for a high Hb to H₂O ratio. Model parameters were otherwise as described for FIGS. 4A-B. Referring to FIG. 4A, a comparison of final photon yield of 840 nm, 1110 nm, 1320 nm, and 1680 nm emitting semiconductor nanocrystals, as a function of tissue scatter and thickness, in tissue with a high H₂O to Hb ratio is shown. Simulated tissues exhibited either wavelength-independent scatter (upper panels) or wavelength-dependent scatter (lower panels). To determine the effect of scatter (left panels), tissue thickness was fixed at 0.5 cm. To determine the effect of tissue thickness, absolute scatter at 630 nm was fixed at 8.9 cm⁻¹ (wavelength-independent scatter) or 23 cm⁻¹ (wavelength-dependent scatter), and the scatter power coefficient fixed at either 0 (wavelength-independent scatter) or 2.81 (wavelength-dependent scatter). On the ordinate is shown the photon yield as a ratio of 1320 nm semiconductor nanocrystals to either 840 nm semiconductor nanocrystals (thick solid line), 1110 nm semiconductor nanocrystals (thin solid line), or 1680 nm semiconductor nanocrystals (dashed line). The simulation described in FIG. 5B was repeated in tissue with a high Hb to H₂O ratio. Note is again made that excitation was broadband, from 400 nm to the peak emission wavelength, using a constant fluence rate at each wavelength. For simplicity, results are displayed as the ratio of the total photon yield of 1320 nm semiconductor nanocrystals relative to the others.

Over the full range of tissue H₂O to Hb ratio, absolute scatter, scatter power coefficient, and thickness tested, 1680 nm semiconductor nanocrystals performed poorly relative to the others, mainly due to the effect of H₂O absorption. In tissues with a high H2O to Hb ratio (FIG. 5A), regardless of scatter power coefficient, 1110 nm semiconductor nanocrystals outperformed 840 nm and 1320 nm semiconductor nanocrystals by up to five-fold. In the presence of wavelength-dependent scatter, 1320 nm semiconductor nanocrystals outperformed 840 nm semiconductor nanocrystals, but only up to two-fold.

In contrast, in the presence of a high Hb to H2O ratio (FIG. 5B), 1320 nm semiconductor nanocrystals outperform 840 nm semiconductor nanocrystals by 32-fold to 5×10³ fold, and 13-fold to 1×10⁶ fold, over the tested range of scatter and thickness, respectively, with the highest performance in tissues with wavelength-dependent scatter. 1320 nm semiconductor nanocrystals also outperform 1110 nm and 1680 nm semiconductor nanocrystals, but by a less significant magnitude. The significance of these results for imaging applications is discussed below.

NIR Fluorescence Imaging of the Coronary Vasculature with Semiconductor Nanocrystals Using Broadband White Light Excitation

Imaging of blood flowing through the coronary vasculature is of paramount importance since even brief ischemia to the myocardium can lead to infarction, and cardiac revascularization requires assessment of vessel patency. An intraoperative NIR fluorescence imaging system that can be used with conventional fluorophores such as indocyanine green and IRDye78 for real-time assessment of coronary vasculature in beating hearts can be used here. See, for example, Nakayama et al., “Functional near-infrared fluorescence imaging for cardiac surgery and targeted gene therapy,” Molecular Imaging (2002), which is incorporated by reference in its entirety. Conventional fluorophores, however, absorb in a relatively narrow wavelength band, and typically require a separate NIR (770 nm) laser light source for excitation. Advantageously, NIR semiconductor nanocrystals can be used in place of conventional fluorophores for vascular imaging, and a single white light source can replace laser excitation, and can be used for both standard illumination and semiconductor nanocrystal fluorescence excitation.

FIGS. 2C, 3, and 4 suggest that for tissues with wavelength-independent scatter and a high Hb to H₂O ratio, such as blood, semiconductor nanocrystals with a peak emission within transmission Band 1 might perform well, provided that tissue thickness is minimal. To choose a semiconductor nanocrystal emission wavelength optimal for the silicon-based CCD camera, the model in spreadsheet format was used to compare semiconductor nanocrystals spanning Band 1. It was determined (data not shown) that semiconductor nanocrystals having peak emission at 752 nm would maximize the number of photons collected by the camera, and these particular NIR semiconductor nanocrystals were synthesized as described above.

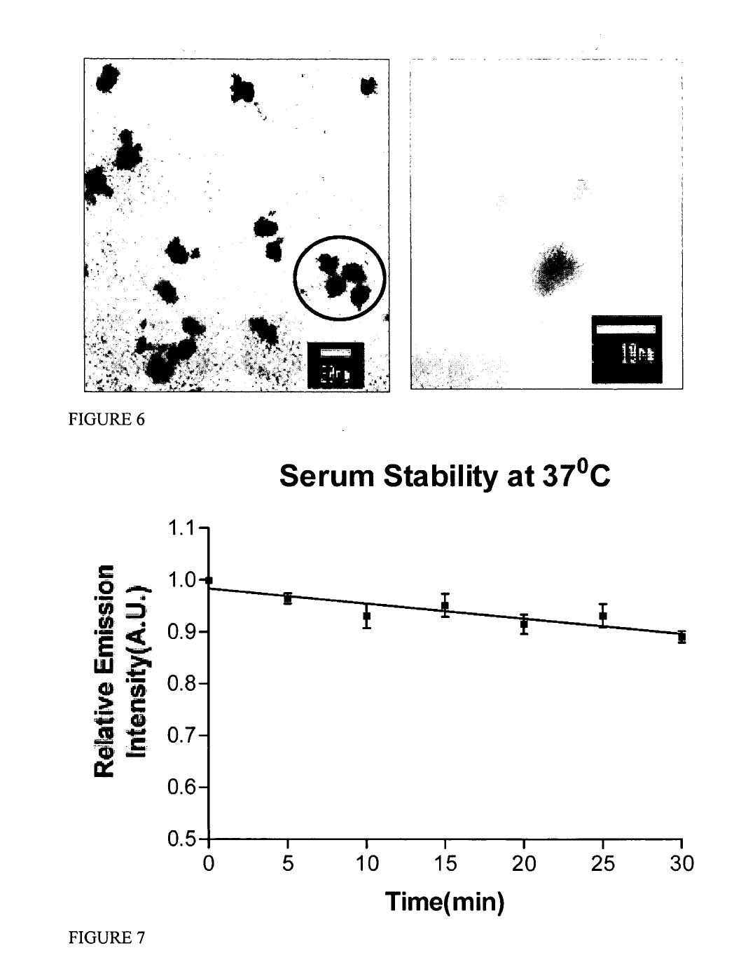

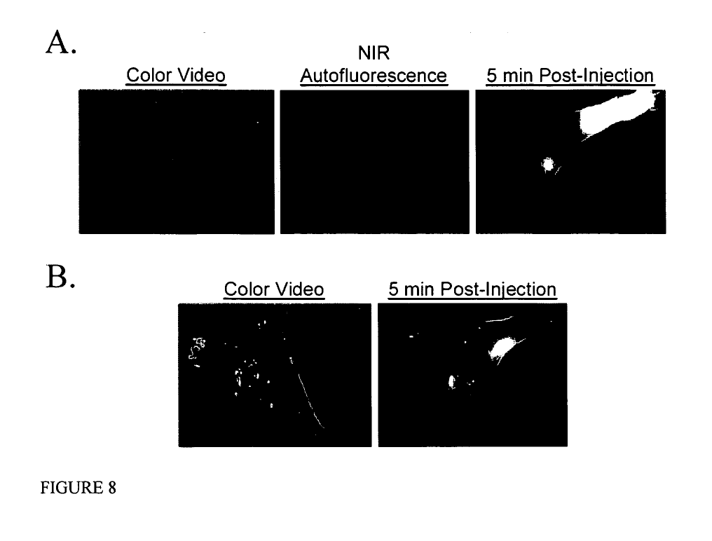

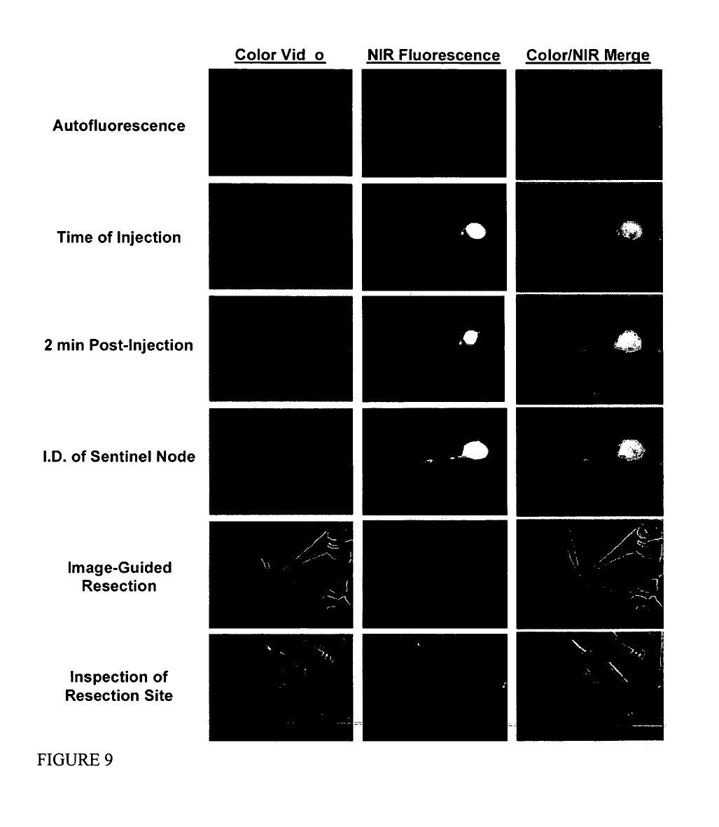

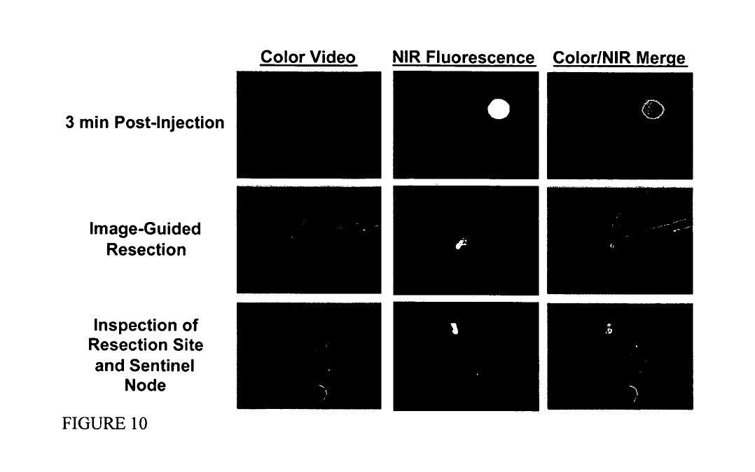

Referring to FIG. 6A, the absorbance (thick black line) and photoluminescence (dashed line) of 752 nm semiconductor nanocrystals embedded in 282 μm of whole blood was simulated using the model described in the text. Shown below the abscissa (black bar) is the broadband wavelength range used for general illumination and semiconductor nanocrystal fluorescence excitation (400 to 700 nm). Referring to FIG. 6B, using the intraoperative NIR fluorescence imaging system, the coronary vasculature of a beating rat heart was imaged before and after intravenous injection of 2.5 nmol of 752 nm emitting semiconductor nanocrystals. Shown are the color video image (upper left panel), pre-injection NIR autofluorescence (upper right panel), post-injection NIR fluorescence (lower left panel) and pseudo-color merged image of the color video and post-injection NIR fluorescence images (lower right panel). Illumination and fluorescence excitation were from the same broadband white light source as shown in FIG. 6A. NIR fluorescence images have identical exposure times (25 msec) and normalization. Shown in FIG. 6A is the simulation of 752 nm semiconductor nanocrystals embedded in arterial blood. Since the average coronary vessel diameter of the rat heart is only 282 μm (see, Szekeres et al., J. Cardiovasc. Pharmacol. 38:584-92 (2001), which is incorporated by reference in its entirety), a 752 nm semiconductor nanocrystals can be excited with broadband white light of 400 to 700 nm, i.e., the same light used to illuminate the surgical field. Indeed, over 75% of absorbed photons would be contained within the 400 nm to 700 nm band. Moreover, the relatively thin coronary vessels of the rat heart were not predicted to degrade emission signal (FIG. 6A). These predictions appear to have been reasonable since intravenous injection of only 2.5 nmol of 752 mn emitting NIR semiconductor nanocrystals into the rat, and broadband white light excitation at a total fluence rate (i.e., the integral of 400 nm to 700 nm light) of only 2.0 mW/cm², resulted in a NIR fluorescence signal of the coronary vasculature with an over 5:1 signal to background ratio for a 25 msec exposure (FIG. 6B). This same signal to noise would require injection of 2.5 nmol of the conventional fluorophore IRDye78-CA and irradiation with a 771 nm laser at a fluence rate of 12.5 mW/cm². See, for example, See, for example, Nakayama et al., “Functional near-infrared fluorescence imaging for cardiac surgery and targeted gene therapy,” Molecular Imaging (2002). These data suggest that, under certain conditions, NIR semiconductor nanocrystals may perform well as in vivo vascular contrast agents using inexpensive white light excitation and a relatively low fluence rate. Intraoperative vascular mapping and angiography of this type can be carried out during all types of human surgery.

The goal of this study was to better understand how tissue absorbance, scatter, and thickness might affect the performance of semiconductor nanocrystals when embedded in biological tissue and used as contrast agents for biomedical assays and imaging. This is based on the assumption that the excitation fluence at the semiconductor nanocrystals is within their linear response regime, and well below their saturation limit. The saturation limit of 840 nm semiconductor nanocrystals is estimated to be on the order of ˜1 kW/cm², and from Fermi's golden rule, ˜0.25 kW/cm² for 1320 nm semiconductor nanocrystals. Indeed, the vascular imaging data was obtained with an external excitation fluence rate of only 2.0 mW/cm².

Biological tissue can have a dramatic filtering effect on semiconductor nanocrystal absorbance (FIGS. 2B-D). Using a previously validated mathematical model that fits well the geometry of reflectance fluorescence imaging, testable hypotheses were formulated regarding the selection of semiconductor nanocrystal wavelengths for biomedical applications. The data suggest that the magnitude of tissue scatter, the scatter power coefficient, tissue thickness, and the ratios of absorbing components can have profound effects on semiconductor nanocrystal excitation and emission wavelength choice. Despite the complexity of a model with many independent variables, several generalizations can be inferred from the data.

In tissues with a high H₂O to Hb ratio and either wavelength-independent scatter (e.g., post-menopausal breast) or wavelength-dependent scatter (e.g., skin), the unique and desired property of semiconductor nanocrystals, namely increasing excitation at bluer wavelengths, is largely preserved (FIG. 4, solid line), and semiconductor nanocrystals emitting in Bands 1, 2, or 3 (FIG. 5A) should perform well, with a slight overall advantage for Band 2.

In tissues with a high Hb to H₂O ratio (e.g., blood), regardless of scatter type, 1320 nm semiconductor nanocrystals outperform 840 nm semiconductor nanocrystals by up to several orders of magnitude over a wide range of tissue thicknesses and absolute values of scatter. Importantly, semiconductor nanocrystal excitation is also often severely constrained to a narrow band very close to the peak emission wavelength (FIG. 4). Hence, under these conditions, the pattern of semiconductor nanocrystal excitation is strikingly similar to that of conventional fluorophores. The emission properties of semiconductor nanocrystals embedded in tissue with wavelength-dependent scatter also differ markedly from non-embedded semiconductor nanocrystals, with a red-shift of peak emission under many conditions.

The results of this study span the extremes of tissue characteristics, from a high Hb to H₂O ratio and wavelength-independent scatter (e.g., blood) to a high H₂O to Hb ratio and wavelength-dependent scatter (e.g., skin). Hence, most tissues will have characteristics between these two extremes. Although the model data suggest that semiconductor nanocrystal excitation and emission wavelengths should be chosen based on the specific tissue(s) being imaged, the data also suggest that Band 3 semiconductor nanocrystals may provide the best overall performance for most biomedical applications. When compared to 840 nm semiconductor nanocrystals, 1320 nm semiconductor nanocrystals are predicted to provide a large improvement in photon yield in tissues such as blood. This result is significant since conventional fluorophores presently being used for biomedical imaging and assays typically have emission within Band 1, i.e., the “near-infrared window” as described in Chance, Ann. N.Y. Acad. Sci. 838:29-45 (1998), which is incorporated by reference in its entirety. For example, Cy7, IRDye78, and indocyanine green emit in the 700 nm to 830 nm range. The results suggest that Band 3 semiconductor nanocrystals may greatly outperform Band 1 semiconductor nanocrystals and conventional fluorophores in many tissues. These improvements may be even more pronounced when considering the typically higher QY of NIR and IR semiconductor nanocrystals over conventional fluorophores and their possible insensitivity to photobleaching. The emission curves for Band 3 semiconductor nanocrystals also fall completely within the sensitivity curve of commercially available indium-gallium-arsenide (InGaAs) cameras, making such imaging practical.

It should be noted that the conclusions of this study are not significantly affected by model geometry. When the simulations were run using an analytical solution to the diffusion equation that utilizes a point light source, rather than uniform irradiance (as described, for example, in Fridolin et al., Phys. Med. Biol. 45:3779-3792 (2000), which is incorporated by reference in its entirety), similar results were obtained. The conclusions also appear to remain valid when single wavelength excitation, rather than broadband excitation, is used. For example, 1320 nm semiconductor nanocrystals are predicted to retain over 65% of their higher photon yield compared to 840 nm semiconductor nanocrystals when both are excited at their respective first excitation peak.

Simulations can result in the design, production and characterization of 752 nm NIR semiconductor nanocrystals specifically tailored for imaging rat coronary vasculature with a silicon CCD camera. The simulation suggested that broadband white light could be used for efficient excitation of such semiconductor nanocrystals (FIG. 6A), and indeed this appears to be the case (FIG. 6B). The ability to predict that inexpensive broadband light sources of low fluence rate can be used in particular applications may help to minimize system engineering and equipment costs.

To simplify the above analysis, the shape of the semiconductor nanocrystal absorbance curves, extinction coefficient at the first absorbance peak, and QY were held constant among the various NIR and IR semiconductor nanocrystals. Of course, the choice of semiconductor material will greatly impact the semiconductor nanocrystal absorbance curve, emission wavelength, particle size, and QY. See, for example, Kershaw et al., IEEE Journal of Selected Topics in Quantum Electronics 6, 534-543 (2000), which is incorporated by reference in its entirety. The shape of the absorbance curve, in particular, will be a strong function of the materials used, and even of the purity of the particular semiconductor nanocrystal preparation. The shape difference between materials such as CdSe (see, for example, Leatherdale et al., J. Phys. Chem. B. 106, 7619-7622 (2002)), CdTe (see, for example, Gaponik et al., J. of Phys. Chem. B 106:7177-7185 (2002), which is incorporated by reference in its entirety), and PbSe (see, for example, Chen et al., Mat. Res .Soc. Symp. Proc. 691:359-364 (2002), which is incorporated by reference in its entirety) had little overall effect when other variables are held constant, and the spreadsheet format of the model made comparative simulation of semiconductor nanocrystal materials straightforward. It should be emphasized that the predictions of the study can be tested immediately. The literature already provides semiconductor material choices and synthetic strategies for semiconductor nanocrystals emitting within Band 1 (CdTe (see, Gaponik et al., J. of Phys. Chem. B 106:7177-7185 (2002) and Murray et al., J. Am. Chem. Soc. 115:8706-8715 (1993), which is incorporated by reference in its entirety) and InP (see, for example, Bruchez et al., Science 281, 2013-2016 (1998)), Band 2 (InAs (Guzelian et al., Applied Physics Letters 69:1432-1434 (1996); and Cao and Banin, J. Am. Chem. Soc. 122:9692-9702 (2000)), Band 3 (HgTe (Rogach et al., Advanced Materials (Weinheim, Germany) 11:552-555 (1999); and Harrison el al., Materials Science & Engineering, B: Solid-State Materials for Advanced Technology B69-70:355-360 (2000), each of which is incorporated by reference in its entirety) and PbSe (Chen et al., Mater. Res. Soc. Symp. Proc. 691:359-364 (2002), which is incorporated by reference in its entirety) and Band 4 (HgTe (Rogach et al., Advanced Materials (Weinheim, Germany) 11:552-555 (1999); and Harrison et al., Materials Science & Engineering, B: Solid-State Materials for Advanced Technology B69-70:355-360 (2000)) and PbSe (Chen et al., Mat. Res .Soc. Symp. Proc. 691:359-364 (2002)).

Simulation can permit semiconductor nanocrystal emission wavelengths to be chosen rationally, before the laborious process of semiconductor nanocrystal production is initiated. Although the absorption advantage of semiconductor nanocrystals can be lost once embedded in certain biological tissue, the tunability of semiconductor nanocrystals to optimal wavelengths remains a feature of paramount importance, and is predicted to result in significant improvements in photon yield over the conventional fluorophores now being used.

Of course, even after choice of optimal excitation and emission wavelengths, it remains to be seen how surface coating, QY, in vivo chemical stability, in vivo photostability, toxicity, and pharmacokinetics will impact the use of semiconductor nanocrystals as contrast agents for biomedical applications. To date, there are no published reports on the toxicity of semiconductor nanocrystals after in vivo administration, and many of the semiconductor materials cited above are known toxins when free in solution. Their toxicity when complexed as nanocrystals remains to be determined. The oligomeric phosphines used for capping in this study appear to have preserved photostability, at least after initial contact with plasma. Clearly the surface coating of semiconductor nanocrystals is of paramount importance with respect to imparting aqueous solubility, minimizing non-specific tissue interactions, and maximizing quantum yield.

Other embodiments are within the scope of the following claims.

1. An imaging composition comprising:
a semiconductor nanocrystal having an outer layer bonded to the nanocrystal.

2. The composition of claim 1, wherein the semiconductor nanocrystal has a diameter of between 5 nm and 10 nm.

3. The composition of claim 1, wherein the outer layer includes a polydentate ligand.

4. The composition of claim 1, wherein the nanocrystal emits light having a wavelength greater than 700 nm.

5. The composition of claim 1, wherein the nanocrystal includes a core of a first semiconductor material and an overcoating of a second semiconductor material on the core wherein the first semiconductor material and the second semiconductor material are selected so that, upon excitation, one carrier is substantially confined to the core and the other carrier is substantially confined to the overcoating.

6. The composition of claim 1, wherein the semiconductor nanocrystal includes a core of a first semiconductor material.

7. The composition of claim 6, wherein the first semiconductor material is a Group II-VI compound, a Group II-V compound, a Group III-VI compound, a Group Ill-V compound, a Group IV-VI compound, a Group I-III-VI compound, a Group II-IV-VI compound, or a Group II-IV-V compound.

8. The composition of claim 6, wherein the first semiconductor material is ZnS, ZnSe, ZnTe, CdS, CdSe, CdTe, HgS, HgSe, HgTe, AIN, AlP, AlAs, AlSb, GaN, GaP, GaAs, GaSb, GaSe, InN, InP, InAs, InSb, TIN, TIP, TIAs, TlSb, PbS, PbSe, PbTe, or mixtures thereof.

9. The composition of claim 6, wherein the semiconductor nanocrystal includes a second semiconductor material overcoated on the first semiconductor material.

10. The composition of claim 9, wherein the first semiconductor material has a first band gap, and the second semiconductor material has a second band gap that is larger than the first band gap.

11. The composition of claim 9, wherein the second semiconductor material is a Group II-VI compound, a Group II-V compound, a Group III-VI compound, a Group III-V compound, a Group IV-VI compound, a Group I-III-VI compound, a Group II-IV-VI compound, or a Group II-IV-V compound.

12. The composition of claim 9, wherein the second semiconductor material is ZnO, ZnS, ZnSe, ZnTe, CdO, CdS, CdSe, CdTe, MgO, MgS, MgSe, MgTe, HgO, HgS, HgSe, HgTe, AIN, AlP, AlAs, AlSb, GaN, GaP, GaAs, GaSb, InN, InP, InAs, InSb, TIN, TIP, TlAs, TlSb, TlSb, PbS, PbSe, PbTe, or mixtures thereof.

13. A method of imaging tissue comprising:
introducing a composition including a semiconductor nanocrystal into the tissue; and
detecting emission from the semiconductor nanocrystal.

14. The method of claim 13, wherein the tissue is vasculature.

15. The method of claim 13, wherein the emission is in the near-infrared (NIR) or infrared wavelength region.

16. The method of claim 13, wherein introducing the composition includes injecting the composition into a body.

17. The method of claim 13, wherein introducing the composition includes injecting the composition into a vascular system of a body.

18. The method of claim 17, wherein detecting emission includes monitoring tissue or tumor vascular during surgery, monitoring body sites of bleeding during surgery, or monitoring tissue perfusion during surgery and surgical repairs.

19. The method of claim 13, wherein the semiconductor nanocrystal has a diameter of between 5 nm and 10 mn.

20. The method of claim 13, wherein the semiconductor nanocrystal has a diameter of between 5 run and 10 mn.

21. The method of claim 13, further comprising exposing the tissue to white light.

22. The method of claim 13, wherein the nanocrystal emits light having a wavelength greater than 700 mn.

23. The method of claim 13, wherein the nanocrystal includes a core of a first semiconductor material and an overcoating of a second semiconductor material on the core wherein the first semiconductor material and the second semiconductor material are selected so that, upon excitation, one carrier is substantially confined to the core and the other carrier is substantially confined to the overcoating.
